(19) United States
(12) Patent Application Publication
(10) Pub. No.: US 2024/0285239 A1
(43) Pub. Date: Aug. 29, 2024

(54) MACHINE SEGMENTATION OF SENSOR MEASUREMENTS AND DERIVATIVES IN VIRTUAL MOTOR EXAMS

(71) Applicant: Verily Life Sciences LLC, South San Francisco, CA (US)

(72) Inventors: Ritu Kapur, Pacifica, CA (US); Maximilien Burq, Fremont, CA (US); Erin Rainaldi, South San Francisco, CA (US); Lance Myers, Pacifica, CA (US); William Marks, San Francisco, CA (US)

(73) Assignee: Verily Life Sciences LLC, South San Francisco, CA (US)

(21) Appl. No.: 18/270,671

(22) PCT Filed: Jan. 31, 2022
(86) PCT No.: PCT/US2022/070435

§ 371 Date: Jun. 30, 2023

Related U.S. Application Data
(60) Provisional application No. 63/200,155, filed on Feb. 17, 2021.

(51) Int. Cl. A61B 5/00; A61B 5/11; G16H 40/67

(52) U.S. Cl. CPC A61B 5/7225; A61B 5/1124; A61B 5/681; G16H 40/67

(57) ABSTRACT

A user device may machine segment sensor measurements by determining a context window. The context window may include a beginning and an end. The context window may be used to define the time period in which measurements of the sensor measurements are to be segmented.

### BACKGROUND

A wearable user device such as a watch may use one or more sensors to sense data representative of physiological signals of a wearer. In some cases, certain sensors may be used (or configured with a different sampling rate) when the wearer performs a predefined action or set of actions requested by the wearable user device. During this time, the sensor data collected may be of varying relevancy to the predefined action or set of actions.

### BRIEF SUMMARY

Various examples are described, including systems, methods, and devices relating to segmenting signal data associated with virtual clinical exams.

A system of one or more computers can be configured to perform particular operations or actions by virtue of having software, firmware, hardware, or a combination of them installed on the system that, in operation, cause the system to perform the actions. One or more computer programs can be configured to perform particular operations or actions by virtue of including instructions that, when executed by data-processing apparatus, cause the apparatus to perform the actions. One general aspect includes a computer-implemented method, which, by utilizing a wearable user device, accesses exam information identifying: (i) a first timing indicator associated with a first time, (ii) a second timing indicator associated with a second time, and (iii) a virtual motor exam type of a virtual motor exam. The method also includes accessing, by the wearable user device, signal data obtained by the wearable user device during a time period bounded by the first time and the second time. The method also includes determining by the wearable user device and based on the virtual motor exam type, a first signal data type for segmenting the signal data as well as first signal data of the first signal data type being output by a first sensor of the wearable user device during the time period. The method also includes determining, by the wearable user device, a context window within the time period by at least selecting a historical signal profile of the first signal data type, the historical signal profile derived from previous occurrences of the virtual motor exam. Determining the context window also includes comparing the first signal data to the historical signal profile to identify a third time corresponding to a beginning of the context window and a fourth time corresponding to an end of the context window of the context window. The method also includes segmenting, by the wearable user device, a portion of the signal data received during the context window. The method also includes generating, by the wearable user device, a virtual motor exam data package based on the portion of the signal data and the exam information. Other embodiments of this aspect include corresponding computer systems, apparatus, and computer programs recorded on one or more computer storage devices, each configured to perform the actions of the methods.

Another general aspect includes a computer-implemented method receiving, at an input device of a wearable user device, a first user input identifying a beginning of a first time period in which a virtual motor exam is conducted. The method also includes receiving, at the input device of the wearable user device, a second user input identifying an end of the first time period. The method also includes accessing, by the wearable user device and based on the virtual motor exam, first signal data output by a first sensor of the wearable user device during the first time period. The method also includes determining, by the wearable user device, a context window within the first time period based on the first signal data and a virtual motor exam type associated with the virtual motor exam, the context window defining a second time period that is within the first time period. The method also includes determining, by the wearable user device, second signal data output by a second sensor of the wearable user device during the second time period. The method also includes associating, by the wearable user device, the second signal data with the virtual motor exam. Other embodiments of this aspect include corresponding computer systems, apparatus, and computer programs recorded on one or more computer storage devices, each configured to perform the actions of the methods.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 illustrates an example system including a wearable device for use in implementing techniques related to segmenting sensor data of virtual motor exams, according to at least one example.

FIG. 2 illustrates a block diagram and a corresponding flowchart illustrating a process for segmenting sensor data of virtual motor exams, according to at least one example.

FIG. 3 illustrates an example flowchart illustrating a process that relates to implementing techniques relating to segmenting sensor data of virtual motor exams, according to at least one example.

FIG. 4 illustrates a diagram including an example sensor and segmented sensor data, according to at least one example.

FIG. 5 illustrates a diagram including example sensors of a single device and segmented sensor data, according to at least one example.

FIG. 6 illustrates a diagram including example sensors of a single device and segmented sensor data, according to at least one example.

FIG. 7 illustrates a detailed view of a portion of the diagram depicted in FIG. 4, according to at least one example.

FIG. 8 illustrates a diagram including example sensors of different devices and segmented sensor data, according to at least one example.

FIG. 9 illustrates an example flowchart illustrating a process related to implementing techniques that relate to segmenting sensor data of virtual motor exams, according to at least one example.

FIG. 10 illustrates an example flowchart illustrating a process related to implementing techniques that relate to segmenting sensor data of virtual motor exams, according to at least one example.

FIG. 11 illustrates an example architecture for implementing techniques related to segmenting sensor data of virtual motor exams, according to at least one example.

### DETAILED DESCRIPTION

Examples are described herein in the context of segmenting sensor data collected by wearable user devices while conducting virtual motor exams on the wearable user devices. Those of ordinary skill in the art will realize that the following description is illustrative only and is not intended to be in any way limiting. For example, the techniques described herein can be used to segment sensor data collected during different types of structured exams, activities, and/or non-structured times, and in some examples, may be implemented on non-wearable user devices. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made to achieve the developer's specific goals, such as compliance with application-and business-related constraints, and that these specific goals will vary from one implementation to another and from one developer to another.

Parkinson's disease and other neurological disorders may cause motor symptoms. Conventionally, a trained clinician will conduct a motor examination at a clinic or in a patient's home to help determine whether the patient's symptoms are related to a certain motor disorder, such as Parkinson's disease, and to also track progression of such disorders. For example, during a physical component such as an exam for Parkinson's, the clinician will look at tremors (e.g., repetitive movement caused by involuntary contractions of muscles), rigidity (e.g., stiffness in arms or legs), bradykinesia or akinesia (e.g., slowness of movement and/or lack of movement during regular tasks), and postural instability (e.g., natural balance issues). In some examples, at least some of the examination may be based on the Unified Parkinson's Disease Rating Scale (UPDRS). Using the wearable device described herein, a patient can conduct these (and other types of) exams at home without physician oversight. The wearable device includes logic to direct the patient's activities, which, in some examples, may require different types of movement or stillness. As described in detail herein, the wearable device includes multiple sensors that collect sensor data as the patient performs these activities. This sensor data can then be processed to derive physiological signals of the patient to identify the same or similar observations as a physician would make during an office visit.

The sensors may be configured to sample at different rates depending on whether an activity is being recorded. In some examples, the patient may indicate when an activity begins and ends. The sensors may collect a large amount of sensor data during this period of time, because of increased sampling rates, increased sampling resolution, increased sensing channels, more active sensors, etc. It has been observed, however, that portions of data gathered during this period of time are more probative of certain key indicators associated with the exam than others. For example, a certain exam may test how well the patient can hold their hands still in their lap while sitting. To begin, the patient may select a “begin” button on a user interface of the wearable device. If the patient is not already sitting, they will need to sit down and move their hands to their lap. During this “preparation” time, the sensors may be sampling a large amount of data, which, in some examples, may not necessarily be probative of how well the patient performs on the current exam. Thus, it is desirable to identify when the data indicates that the patient has finished their preparation and is actually performing the exam. The techniques described herein are adapted to determine this actual beginning time and an actual ending time. The window of time in which the patient is actually performing the exam may be referred to herein as a context window, having a beginning and an end. The context window can be determined using a historical signal profile for a first type of sensor and sensor data collected by a sensor of the first type (e.g., first sensor measurements and derivatives). Once the context window has been defined using sensor data from one sensor (or more sensors, in some examples), the context window can be used to machine segment portions of sensor data that are most important for the exam and that were collected by various sensors of the device. Machine segmentation may include segmenting the sensor data in an automated manner. Other sensor data collected before and after the context window may be disregarded or at least given less weight in the analysis of how well the patient performed on the exam. Similar techniques can be applied to sensors housed on different devices that also may not be time-synced with the sensors on the wearable device. In addition, applying the techniques described herein to sensors on different devices may enable time alignment between sensor data collected using the other devices and the wearable device.

In a particular example, a patient is provided a wearable device such as a watch as part of a disease progression program. The watch may include a set of sensors configured to track various movements, heart rate, etc. of the patient and software to conduct various virtual motor exams. The virtual motor exams may be accessed on demand by the user and/or the watch may suggest a suitable time for conducting an exam. In either case, to begin an exam, the patient may select a button (e.g., a physical button or graphical user interface (“GUI”) element) and the same or a different button to end. The wearable device may generate timestamps to indicate the beginning and the end of the exam, which may be associated with an exam identifier (e.g., an identifier that uniquely identifies the type of exam) and a session identifier (e.g., an identifier that uniquely identifies a session in which the exam was conducted). Additionally, during the exam, the wearable device may instruct multiple sensors to collect sensor data, which may be obtained in the form of sensor measurements and derivatives and/or user inputted data. The sensor measurements and other collected sensor data may take the form of signal data. After the exam has concluded or during the exam, using the techniques described herein, the wearable device may determine a context window that represents some period of time during the exam in which the signal data is representative of the patient performing the relevant activities of the exam. To do so, the wearable device selects sensor data collected during the exam from a first sensor and compares the sensor data to a historical signal profile for the exam type and the same type of sensor. The results of this comparison, which can be performed heuristically and/or using a machine-learning model, may include a beginning of the context window and, in some examples, an end of the context window. The beginning and end of the context window may be represented as timestamps, which can then be used to segment out portions of sensor data from the first sensor and output from other sensors of the wearable device. Once the sensor data has been segmented, the wearable device generates a virtual motor exam data package using the segmented signal data and information about the exam. This virtual motor exam data package may be shared with a remote server for further processing and/or be shared with the patient's physician. In some examples, the output can also be used to adjust the operation of sensors during future exams.

This illustrative example is given to introduce the reader to the general subject matter discussed herein, and the disclosure is not limited to this example. The following sections describe various additional non-limiting examples of techniques relating to segmenting signal data collected during virtual motor exams.

The techniques described herein enable one or more technical improvements to the computers that implement virtual motor exams. For example, battery power of portable user devices may be conserved because sensor sampling rates on future exams may be adjusted based on information learned during an analyzed exam. Additionally, the approaches described herein can improve the probative value of the data collected during an exam, and because the context window can be determined by output from one sensor and applied to output from all other sensors, the tedious and processor-intensive process of post-processing signal alignment of all sensor data is avoided. This approach conserves computing resources on the user devices, which allows these resources to be used for other purposes such as processing sensor data, updated user interfaces, and the like. Patient privacy is also conserved because, rather than sending the sensor data to a remote server for processing, the data is processed by the wearable device at least until a data package is generated, which can be encrypted and shared with the remote server in a secure manner.

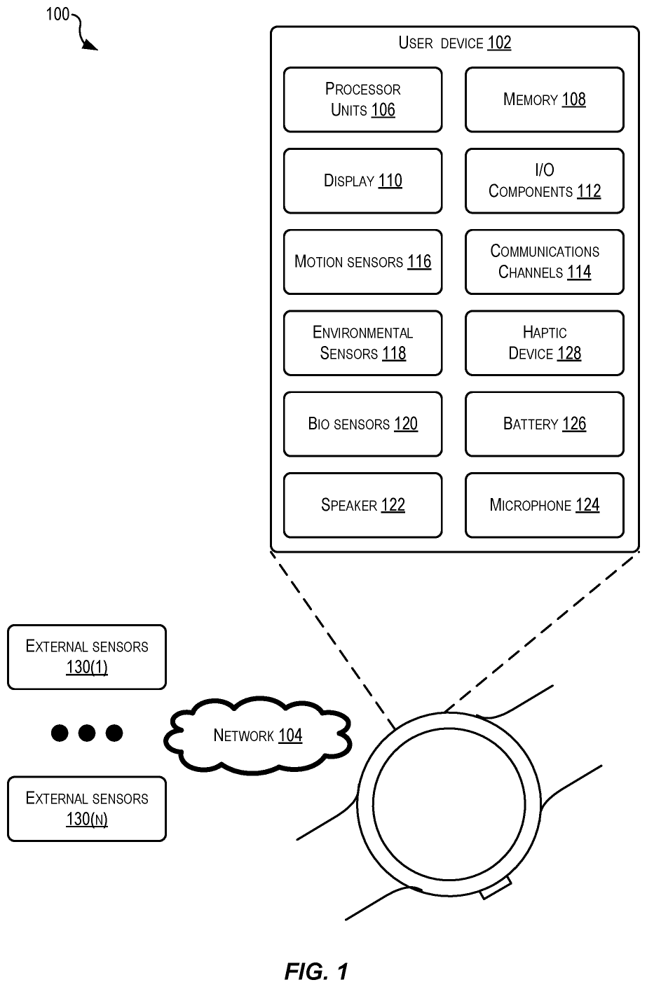

Turning now to the figures, FIG. 1 illustrates an example system including a user device for use in implementing techniques related to segmenting sensor data of virtual motor exams, according to at least one example. The system 100 includes a user device 102 such as wearable user device that may communicate with various other devices and systems via one or more networks 104.

Examples described herein may take the form of, be incorporated in, or operate with a suitable wearable electronic device such as, for example, a device that may be worn on a user's wrist and secured thereto by a band. The device may have a variety of functions, including, but not limited to: keeping time; monitoring a user's physiological signals and providing health-related information based at least in part on those signals; communicating (in a wired or wireless fashion) with other electronic devices, which may be different types of devices having different functionalities; providing alerts to a user, which may include audio, haptic, visual, and/or other sensory output, any or all of which may be synchronized with one another; visually depicting data on a display; gathering data from one or more sensors that may be used to initiate, control, or modify operations of the device; determining a location of a touch on a surface of the device and/or an amount of force exerted on the device, and using either or both as input; accepting voice input to control one or more functions; accepting tactile input to control one or more functions; and so on.

As shown in FIG. 1, the user device 102 includes one or more processor units 106 that are configured to access a memory 108 having instructions stored thereon. The processor units 106 of FIG. 1 may be implemented as any electronic device capable of processing, receiving, or transmitting data or instructions. For example, the processor units 106 may include one or more of: a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitably configured computing element or elements.

The memory 108 may include removable and/or non-removable elements, both of which are examples of non-transitory computer-readable storage media. For example, non-transitory computer-readable storage media may include volatile or non-volatile, removable or non-removable media implemented in any method or technology for storage of information such as computer-readable instructions, data structures, program modules, or other data. The memory 108 is an example of non-transitory computer storage media. Additional types of computer storage media that may be present in the user device 102 may include, but are not limited to, phase-change RAM (PRAM), static random-access memory (SRAM), dynamic random-access memory (DRAM), random-access memory (RAM), read-only memory (ROM), electrically erasable programmable read-only memory (EEPROM), flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the user device 102. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media.

In addition to storing computer-executable instructions, the memory 108 may be configured to store historical sensor data profiles. A historical sensor data profile may identify, for a particular type of exam, configuration settings for operating the sensors of the user device 102. In some examples, the historical sensor data profile may be generated using historical data collected from other occurrences of the exam conducted for other users in a controlled or uncontrolled environment. Machine-learning techniques may be applied to the historical data to build the profile. The historical sensor data profile may include a tagged beginning of the actual exam and a tagged ending of the actual exam, which may be different from the beginning and end identified by the users. The historical data profile may be received by the user device 102 from a server computer or other external device that has access to sensor data for multiple users.

The instructions or computer programs may be configured to perform one or more of the operations or functions described with respect to the user device 102. For example, the instructions may be configured to control or coordinate the operation of the various components of the device. Such components include, but are not limited to, display 110, one or more input/output (I/O) components 112, one or more communication channels 114, one or more motion sensors 116, one or more environmental sensors 118, one or more bio sensors 120, a speaker 122, microphone 124, a battery 126, and/or one or more haptic feedback devices 128.

The display 110 may be configured to display information via one or more graphical user interfaces and may also function as a input component, e.g., as a touchscreen. Messages relating to the execution of exams may be presented at the display 110 using the processor units 106.

The I/O components 112 may include a touchscreen display, as described, and may also include one or more physical buttons, knobs, and the like disposed at any suitable location with respect to a bezel of the user device 102. In some examples, the I/O components 112 may be located on a band of the user device 102.

The communication channels 114 may include one or more antennas and/or one or more network radios to enable communication between the user device 102 and other electronic devices such as one or more other external sensors 130, other electronic devices such as a smartphone or tablet, other wearable electronic devices, external computing systems such as a desktop computer or network-connected server. In some examples, the communication channels 114 may enable the user device 102 to pair with a primary device such as a smartphone. The pairing may be via Bluetooth or Bluetooth Low Energy (“BLE”), near-field communication (“NFC”), or other suitable network protocol, and may enable some persistent data sharing. For example, data from the user device 102 may be streamed and/or shared periodically with the smartphone, and the smartphone may process the data and/or share with a server. In some examples, the user device 102 may be configured to communicate directly with the server via any suitable network, e.g., the Internet, a cellular network, etc.

The sensors of the user device 102 may be generally organized into three categories including motion sensors 116, environmental sensors 118, and bio sensors 120. As described herein, reference to “a sensor” or “sensors” may include one or more sensors from any one and/or more than one of the three categories of sensors. In some examples, the sensors may be implemented as hardware elements and/or in software.

Generally, the motion sensors 116 may be configured to measure acceleration forces and rotational forces along three axes. Examples of motion sensors include accelerometers, gravity sensors, gyroscopes, rotational vector sensors, significant motion sensors, step counter sensor, Global Positioning System (GPS) sensors, and/or any other suitable sensors. Motion sensors may be useful for monitoring device movement, such as tilt, shake, rotation, or swing. The movement may be a reflection of direct user input (for example, a user steering a car in a game or a user controlling a ball in a game), but it can also be a reflection of the physical environment in which the device is sitting (for example, moving with a driver in a car). In the first case, the motion sensors may monitor motion relative to the device's frame of reference or your application's frame of reference; in the second case the motion sensors may monitor motion relative to the world's frame of reference. Motion sensors by themselves are not typically used to monitor device position, but they can be used with other sensors, such as the geomagnetic field sensor, to determine a device's position relative to the world's frame of reference. The motion sensors 116 may return multi-dimensional arrays of sensor values for each event when the sensor is active. For example, during a single sensor event the accelerometer may return acceleration force data for the three coordinate axes, and the gyroscope may return rate of rotation data for the three coordinate axes.

Generally, the environmental sensors 118 may be configured to measure environmental parameters such as temperature and pressure, illumination, and humidity. The environmental sensors 118 may also be configured to measure physical position of the device. Examples of environmental sensors 118 may include barometers, photometers, thermometers, orientation sensors, magnetometers, Global Positioning System (GPS) sensors, and any other suitable sensor. The environmental sensors 118 may be used to monitor relative ambient humidity, illuminance, ambient pressure, and ambient temperature near the user device 102. In some examples, the environmental sensors 118 may return a multi-dimensional array of sensor values for each sensor event or may return a single sensor value for each data event. For example, the temperature in ° C. or the pressure in hPa. Also, unlike motion sensors 116 and bio sensors 120, which may require high-pass or low-pass filtering, the environmental sensors 118 may not typically require any data filtering or data processing.

The environmental sensors 118 may also be useful for determining a device's physical position in the world's frame of reference. For example, a geomagnetic field sensor may be used in combination with an accelerometer to determine the user device's 102 position relative to the magnetic north pole. These sensors may also be used to determine the user device's 102 orientation in some of frame of reference (e.g., within a software application). The geomagnetic field sensor and accelerometer may return multi-dimensional arrays of sensor values for each sensor event. For example, the geomagnetic field sensor may provide geomagnetic field strength values for each of the three coordinate axes during a single sensor event. Likewise, the accelerometer sensor may measure the acceleration applied to the user device 102 during a sensor event. The proximity sensor may provide a single value for each sensor event.

Generally, the bio sensors 120 may be configured to measure biometric signals of a wearer of the user device 102 such as, for example, heart rate, blood oxygen levels, perspiration, skin temperature, etc. Examples of bio sensors 120 may include a heart rate sensor (e.g., photoplethysmography (PPG) sensor, electrocardiogram (ECG) sensor, electroencephalography (EEG) sensor, etc.), pulse oximeter, moisture sensor, thermometer, and any other suitable sensor. The bio sensors 120 may return multi-dimensional arrays of sensor values and/or may return single values, depending on the sensor.

The acoustical elements, e.g., the speaker 122 and the microphone 124 may share a port in housing of the user device 102 or may include dedicated ports. The speaker 122 may include drive electronics or circuitry and may be configured to produce an audible sound or acoustic signal in response to a command or input. Similarly, the microphone 124 may also include drive electronics or circuitry and is configured to receive an audible sound or acoustic signal in response to a command or input. The speaker 122 and the microphone 124 may be acoustically coupled to a port or opening in the case that allows acoustic energy to pass, but may prevent the ingress of liquid and other debris.

The battery 126 may include any suitable device to provide power to the user device 102. In some examples, the battery 126 may be rechargeable or may be single use. In some examples, the battery 126 may be configured for contactless (e.g., over the air) charging or near-field charging.

The haptic device 128 may be configured to provide haptic feedback to a wearer of the user device 102. For example, alerts, instructions, and the like may be conveyed to the wearer using the speaker 122, the display 110, and/or the haptic device 128.

The external sensors 130(1)-130(n) may be any suitable sensor such as the motion sensors 116, environmental sensors 118, and/or the bio sensors 120 embodied in any suitable device. For example, the sensors 130 may be incorporated into other user devices, which may be single or multi-purpose. For example, a heart rate sensor may be incorporated into a chest band that is used to capture heart rate data at the same time as the user device 102 captures sensor data. In other examples, position sensors may be incorporated into devices and worn at different locations on a human user. In this example, the position sensors may be used to track positional location of body parts (e.g., hands, arms, legs, feet, head, torso, etc.). Any of the sensor data obtained from the external sensors 130 may be used to implement the techniques described herein.

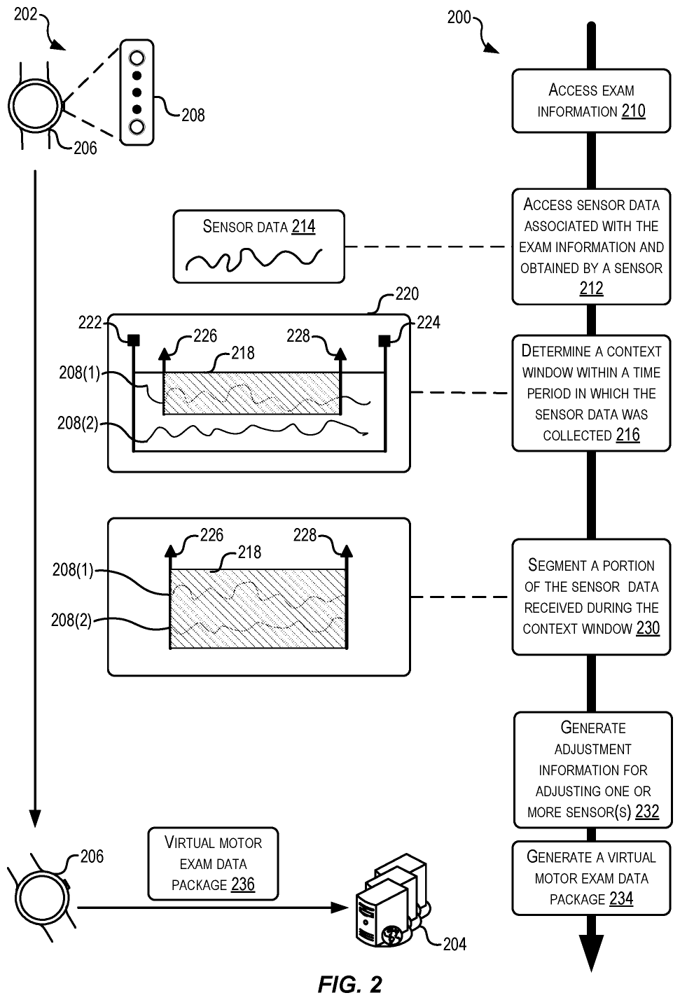

FIG. 2 illustrates a system 202 and a corresponding flowchart illustrating a process 200 for segmenting sensor data of virtual motor exams, according to at least one example. The system 202 includes a service provider 204 and a user device 206. FIG. 2 illustrates certain operations taken by the user device 206 as it relates to information segmenting sensor data. The user device 206 is an example of the user device 102 introduced previously.

As described in further detail herein, the service provider 204 may be any suitable computing device (e.g., personal computer, handheld device, server computer, server cluster, virtual computer) configured to execute computer-executable instructions to perform operations such as those described herein. The computing devices may be remote from the user device 206. The user device 206, as described herein, is any suitable portable electronic device (e.g., wearable device, handheld device, implantable device) configured to execute computer-executable instructions to perform operations such as those described herein. The user device 206 includes one or more onboard sensors 208. The sensors 208 are examples of the sensors 116-120 described herein.

The service provider 204 and the user device 206 may be in network communication via any suitable network such as the Internet, a cellular network, and the like. In some examples, the user device 206 may be intermittently in network communication with the service provider 204. For example, the network communications may be enabled to transfer data (e.g., virtual exam data packages, adjustment information) which can be used by the service provider 204 for sharing with relevant parties and for improving exam administration on the user device 206 and other user devices 206. In some examples, the user device 206 is in network communication with the service provider 204 via a primary device. For example, the user device 206, as illustrated, may be a wearable device such as a watch. In this example, the primary device may be a smartphone that connects to the wearable device via a first network connection (e.g., Bluetooth) and connects to the service provider 204 via a second network connection (e.g., cellular). In some examples, however, the user device 206 may include suitable components to enable the user device 206 to communicate directly with the service provider 204.

The process 200 illustrated in FIG. 2 provides an overview of how the system 202 may be employed to segment sensor data of virtual motor exams. The process 200 may begin at block 210 by the user device 206 accessing exam information. The exam information may be generated by the user device 206 during or after a virtual motor exam has been conducted. The exam information may indicate characteristics of a virtual motor exam, such as a type of virtual motor exam, a task associated with the type of virtual motor exam, user-or system-provided timestamps identifying a beginning and an end of the exam, user-provided feedback about the exam, and other information about the exam. In some examples, the user device 206 accesses the exam information from a memory of the user device 206.

At block 212, the user device 206 accesses sensor data 214 associated with the exam information and obtained by a sensor 208(1) (e.g., one of the sensors 208). In some examples, the sensor data 214 may have been collected during the exam identified by the exam information accessed at block 210. In some examples, the sensor data 214 may be processed by the sensor that generates the sensor data 214 (e.g., filters, digitizes, packetizes, etc.). In some examples, the sensors 208 provide the sensor data 214 without any processing. Logic on the user device 206 may control the operation of the sensors 208 as it relates to data collection during the exam. All of the sensors 208 may be time-aligned because they are all on the same device (e.g., the user device 206) and thereby aligned with the same internal clock (e.g., a clock of the user device 206). If not, the techniques described herein can be used to time-align sensor data in addition to segmenting sensor data.

At block 216, the user device 206 determines a context window 218 within a time period in which the sensor data was collected. As illustrated in exam information block 220, an exam may include a beginning 222 and an end 224 as indicated by the exam information accessed at block 210, and sensor data output from two or more sensors 208(1) and 208(2) obtained at least in part at block 212. Block 216 includes using the sensor data output from the sensor 208(1) to determine a beginning 226 of the context window and, in some examples, an end 228 of the context window 218. Various approaches are described herein for performing the block 216. In some examples, this may include comparing the sensor data output by the sensor 208(1) with a historical signal profile for sensor data output by the sensor 208(1).

At block 230, the user device 206 segments a portion of the signal data received during the context window 218. This may include using the beginning 226 and the end 228 to define a period of time including sensor data of greater interest than, perhaps, data obtained outside of the context window 218 (e.g., between the beginning 222 and the beginning 226 and between the end 228 and the end 224). Segmenting the portion of the signal data may include using the context window 218 not only to segment the data output by the sensor 208(1), but also to segment the data output by the sensor 208(2). In this manner, the context window determined using sensor data output by one sensor 208 of the user device 206 can be applied to sensor data output by any number of other sensors 208 of the user device 206.

At block 232, the user device 206 generates adjustment information for adjusting one or more sensors 208. This may include using the determined beginning 226 and end 228 as control points for determining when to adjust sampling rates and/or turn on and off the sensors 208 during future exams (e.g., future sensor events). The adjustment information may include control information for controlling the operation of the one or more sensors 208 during the future sensor events. In some examples, the adjustment information includes updated parameter values of the sensors 208. This may include offsets, calibrations, and the like. In some examples, the process 200 may also include the user device 206 using the adjustment information to adjust the sensors 208.

At block 234, the user device 206 generates a virtual motor exam data package 236. This may include the exam information, the segmented sensor data, and, in some examples, the context window. In some examples, the virtual motor exam data package 236 may include other information relating to the virtual motor exam. For example, images, videos, text, and the like may be bundled with the virtual motor exam data package. In some examples, the segmented sensor data and the information that defines the context window may be identified by the user device 206, as described herein, and shared with the service provider 204 via a network such as the network 104. The virtual motor exam data package 236 may be useable by the user device 206 and/or the service provider 204 to assess how the user performed on the exam. In some examples, the service provider 204 may share aspects of the virtual exam data package 236 with other users such as medical professionals who are monitoring managing the virtual exam.

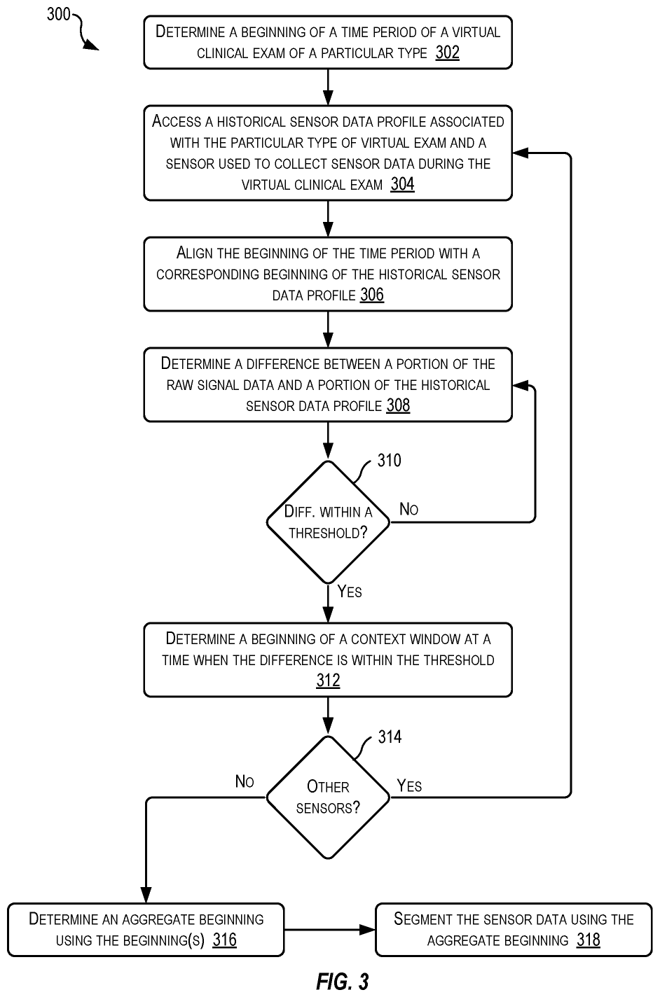

FIGS. 3, 9, and 10 illustrate example flow diagrams showing processes 300, 900, and 1000 according to at least a few examples. These processes and any other processes described herein (e.g., the process 200) are illustrated as logical flow diagrams, each operation of which represents a sequence of operations that can be implemented in hardware, computer instructions, or a combination thereof. In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, one or more applications) executing collectively on one or more processors, by hardware, or combinations thereof. As noted above, the code may be stored on a non-transitory computer-readable storage medium, for example, in the form of a computer program including a plurality of instructions executable by one or more processors.

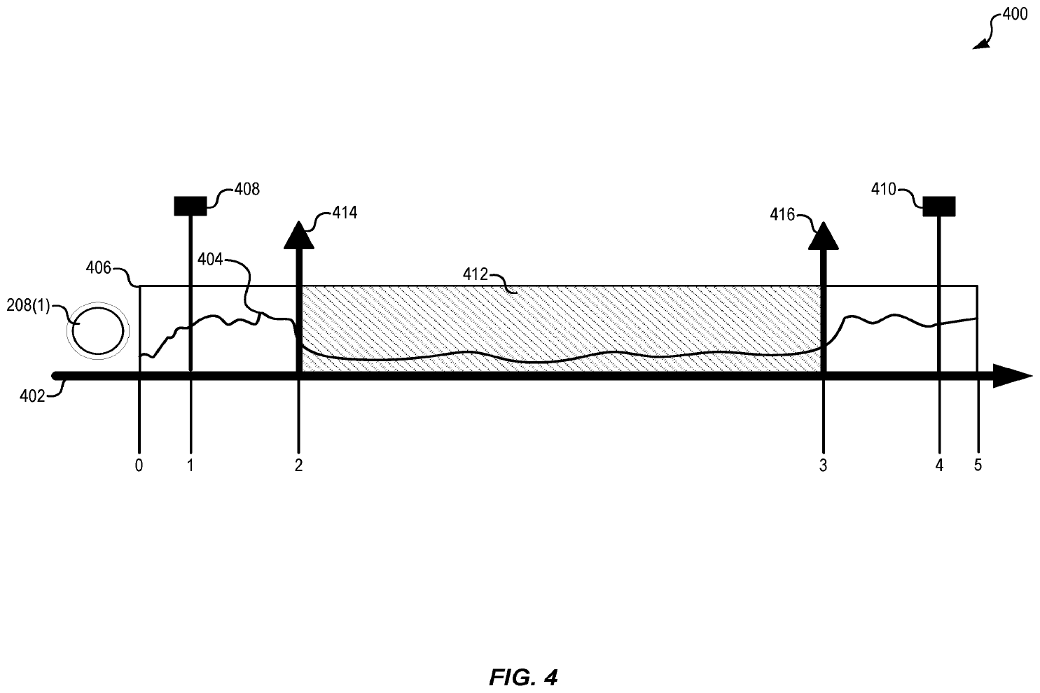

FIG. 3 illustrates an example flowchart illustrating the process 300 relating to implementing techniques relating to segmenting sensor data of virtual motor exams, according to at least one example. FIGS. 4-7 illustrate diagrams including various example sensors, example segmented sensor data, and various devices. FIGS. 4-7 will be introduced with respect to FIG. 3. The process 300 is performed by the user device 102 (FIG. 1). The process 300 in particular corresponds to various approaches for segmenting sensor data, according to various examples.

The process 300 begins at block 302 by the user device 102 determining a beginning of a time period of a virtual clinical exam of a particular type. This may include determining the beginning based on exam information such as described in FIGS. 2 at 210, 212, and 216. This may include using timestamps corresponding to user inputs at the user device 206.

At block 304, the process 300 includes the user device 102 accessing a historical sensor data profile associated with the particular type of virtual exam and a sensor used to collect sensor data during the virtual clinical exam. This may include the user device 102 using a set of evaluation rules to determine which historical sensor data profile is appropriate. In some examples, the historical sensor data profile may be accessed from memory of the user device 102 and/or requested from an external computing system. The evaluation rules may define, for a particular exam type, which profile is appropriate. The historical sensor data profile may be specific to a type of exam (e.g., sit and stand, hand movement, balance on one foot) and be specific to a type of sensor (e.g., accelerometer, gyroscope, heart rate monitor, etc.).

At block 306, the process 300 includes the user device 102 aligning the beginning of the time period with a corresponding beginning of the historical sensor data profile. This may include using the timestamped time of the beginning of the time period (e.g., when the user requested the exam to begin or otherwise interacted with the user device 102) and a corresponding time in the historical sensor data profile. In some examples, the sensor data profile may have one or more other alignment points to ensure proper alignment with the time period. For example, certain values (e.g., highest and/or lowest values) in the historical data profile may be tagged as alignment points and matched to highest and/or lowest values in the sensor data.

At block 308, the process 300 includes the user device 102 determining a difference between a portion of the signal data and a portion of the historical sensor data profile. As it can be assumed that the signal data will not be a perfect match with the historical sensor data profile, the user device 102 can compare the two to identify location(s) where the differences are minor and/or major, depending on a set of evaluation rules, or an overall similarity between the different profiles, e.g., based on an average difference between corresponding data points being below a threshold. For example, the evaluation rules may indicate that for a particular exam type and for this particular sensor, the user device 102 should expect to see large signal fluctuations during a “preparation time” and low signal fluctuations during the actual test. The historical signal profile may represent an averaged or learned depiction of these fluctuations.

At block 310, the process 300 includes the user device 102 using the historical signal profile to determine whether the differences are within some threshold. Small differences may indicate that the portion of the signal data is aligning with the historical signal profile. If the differences are too great, then the process 300 may return to the block 308 to continue to determine differences. If the differences are within the threshold, the process 300 may continue to block 312.

At block 312, the process 300 includes the user device 102 determining a beginning of the context window at a time when the difference is within the threshold. In this example, the beginning from the historical signal profile may be set as the beginning of the context window because the difference at that point is within the threshold. In some examples, other locations along the historical signal profile may be compared to identify the end of the context window and/or a different beginning. A similar threshold comparison may be performed at these select locations to determine whether the other points are aligned with the historical signal profile. For example, the sensor data may be divided up into some equivalent chunks of time (e.g., 10), and sensor values at the beginning of each chunk may be compared to corresponding values at the same time in the historical profile. The difference between values at these points may be compared to determine whether the end of the context window is likely within one of the chunks. When this is determined, that chunk may be divided into yet smaller chunks, and the process may be repeated to identify similarities over this shorter period of time. In some examples, the threshold difference may be smaller for the second time through the process. This approach may be repeated consecutively a fixed number of times, until some aggregated differences in the values is less than some difference threshold, or in any other suitable manner.

At block 314, the process 300 includes the user device 102 determining whether there are other sensors that can be used to determine a different context window. If so, the process 300 returns to the block 304 and accesses a different historical sensor data profile associated with the particular type of virtual exam and a different sensor that collects different sensor data during the clinical exam. Once the different context window has been generated, the process 300 proceeds to block 316, at which the process 300 includes the user device 102 determining an aggregate beginning using the beginning of the context window alone or the beginning of the context window together with the beginnings of one or more different context windows. In some examples, determining the aggregate beginning may include defining the aggregate beginning as the same time as the beginning determined at 312. In some examples, determining the aggregate beginning may include picking the aggregate beginning from among a set of beginnings including the beginning of the context window and the beginnings of the one or more different context windows. For example, such picking may be based on the sensor type used to define the context window and/or the exam type. This may be possible because data output by certain sensors may be more reliable for determining the beginning of the context window than others and/or certain exam types may have better defined beginnings (and ends) than others exam types. In some examples, determining the aggregate beginning may include averaging the times for the beginning and the other beginnings.

At block 318, the process 300 includes the user device 102 using the aggregate beginning to segment a portion of the sensor data collected by the user device 102. As described in FIG. 2, this may also be used to generate the data package and/or adjustment information.

FIG. 4 illustrates a diagram 400 including an example sensor 208(1) and segmented sensor data, according to at least one example. The diagram 400 is an example of using a single sensor to segment data for that particular sensor. The diagram 400 includes a timeline 402 and a representation of sensor data 404 obtained by the sensor 208(1) (e.g., one of the sensors 116-120) during some period 406, extending between t=0 and t=5. A profile of the sensor data 404 may vary at different times between t=0 and t=5. In some examples, the sampling rate of the sensor may vary between t=0 and t=5. The diagram 400 also includes timestamps 408 and 410 corresponding, respectively, to a user input or a machine-defined beginning and end of a virtual motor exam. For example, after being prompted by the user device 102 to perform the virtual exam, the user may, at t=1, provide a command to the user device 102 in the form of a user interface selection, physical button, voice command, etc. to begin the virtual motor exam. The timestamp 408 may be generated by the user device 206 and associated with the sensor data 404 at that time. This may correspond to the block 302 of FIG. 3.

Between t=1 and t=2, the user may still be preparing for the exam (e.g., getting situated, assuming some predefined position). Because of this, the data collected between t=1 and t=2 may be less relevant to the actual results of the virtual motor exam. Thus, the blocks 304-312 may be performed to identify a context window 412 bounded by a window beginning 414 and a window end 416. As can be seen in the example sensor data 404, the window beginning 414 and the window end 416 may correspond to inflection points in the data or other locations where a larger change in slope is observed. For example, between t=2 and t=3, the user may be doing their best to perform the virtual motor exam, for example, by sitting still and holding their hands in their lap. Thus, during this time, the sensor 208(1) (e.g., an accelerometer) shows very little movement. But as the virtual motor exam ends, the sensor data 404 shows more variation (e.g., between t=3 and t=4). In some examples, the window end 416 is not a determined value, but rather it is matched to the end 410, which may be user-defined by selecting inputting at the user device that the exam has concluded or the end 410 may be auto-defined (e.g., the virtual exam may run for a fixed period and may automatically end after the time has elapsed). The portion of the sensor data 404 within the context window 412 may be segmented from the other sensor data 404 and stored together with other information about the virtual motor exam (e.g., exam type, sensor type, window beginning, window end), as described in block 318.

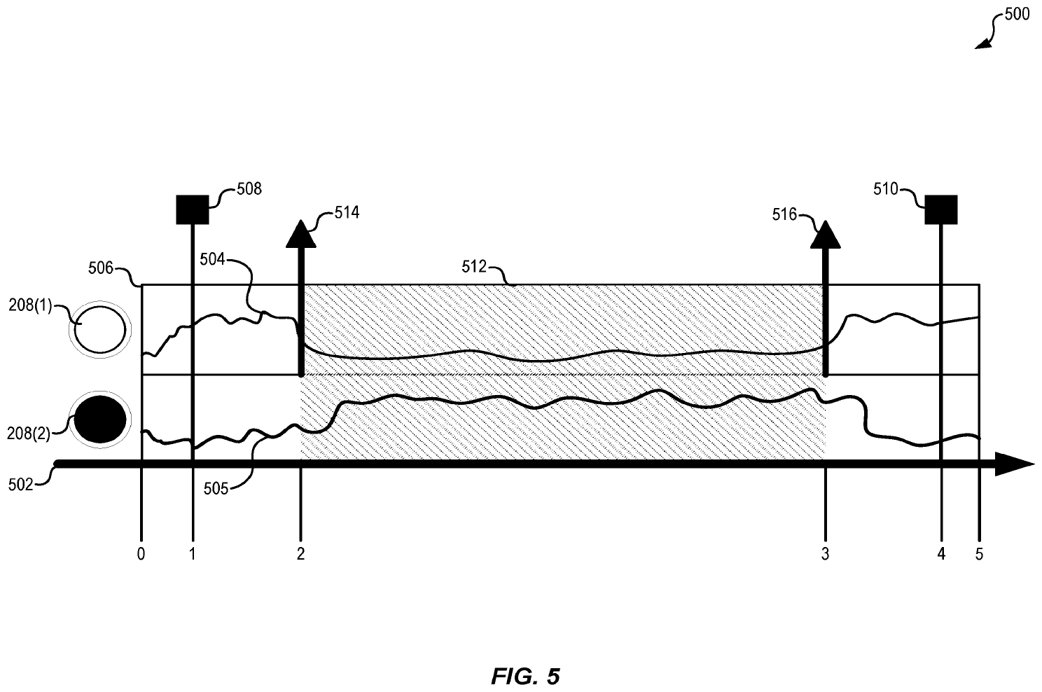

FIG. 5 illustrates a diagram 500 including example sensors 208(1) and 208(2) of the same user device 102 and segmented sensor data, according to at least one example. The diagram 500 is an example of using a first sensor 208(1) to segment data for that particular sensor and data obtained from a second sensor 208(2). The diagram 500 includes a timeline 502 and a representation of first sensor data 504 obtained by the first sensor 208(1) and sensor data 505 obtained by the second sensor 208(2), all during some period 506, extending between t=0 and t=5. Profiles of the sensor data 504 and 505 may vary at different times between t=0 and t=5. In some examples, the sampling rates of the sensors 208(1) and 208(2) may vary between t=0 and t=5. The diagram 500 also includes timestamps 508 and 510 corresponding, respectively, to a user input or a machine-defined beginning and end of a virtual motor exam. For example, after being prompted by the user device 102 to perform the virtual motor exam, the user may, at t=1, select a button on the user device 102 to begin the virtual motor exam. The timestamp 508 may be generated by the user device 102 and associated with the sensor data 504 and 505 at that time. This may correspond to the block 302 of FIG. 3. In FIG. 5, a context window 512 bounded by a window beginning 514 and a window end 516 may be determined similar as described with respect to context window 412 of FIG. 4. The difference in FIG. 5, however, is in the segmenting step. In this example, the sensor data 504 is used to generate the context window 512, and the context window 512 is used to segment the sensor data 504 and the sensor data 505 (obtained by the sensor 208(2)). The portion of the sensor data 504 and 505 within the context window 512 may be segmented from the other sensor data 504 and 505 and stored together with other information about the virtual motor exam (e.g., exam type, sensor type, window beginning, window end), as described in block 318.

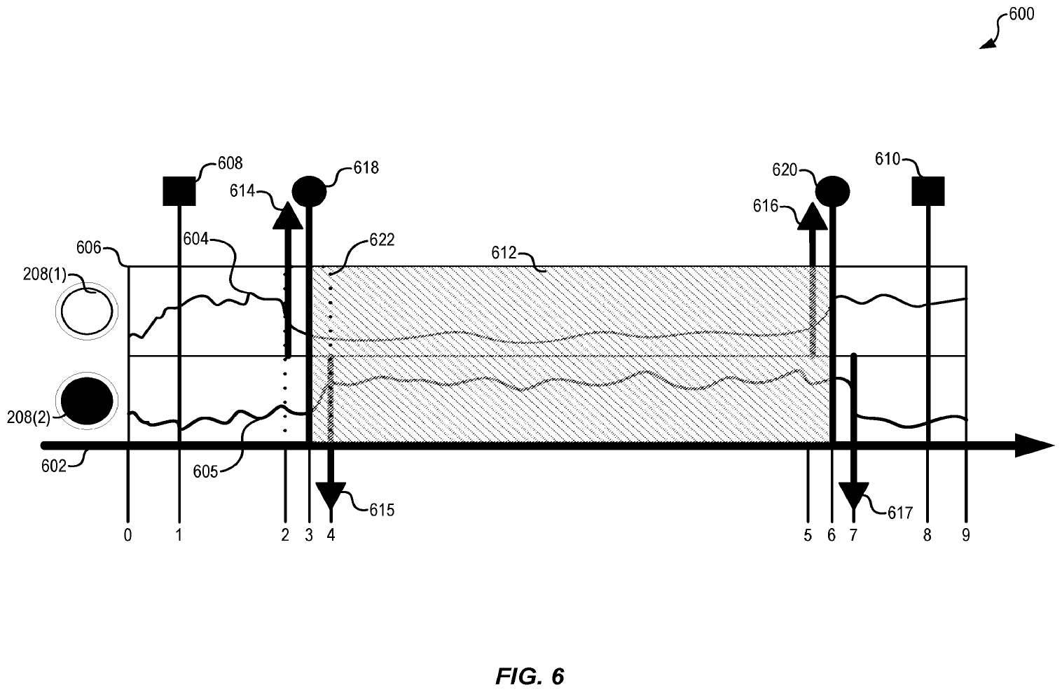

FIG. 6 illustrates a diagram 600 including example sensors 208(1) and 208(2) of the same user device 102 and segmented sensor data, according to at least one example. The diagram 600 is an example of using a first sensor 208(1) to determine a first window beginning 614 for a context window 612 and using a second sensor 208(2) to determine a second window beginning 615 for the context window 612. These different beginnings 614 and 615 can be used to generate an aggregate beginning 618. First and second window ends 616 and 617 can also be used to generate an aggregate end 620, as described herein. The diagram 600 includes a timeline 602 and a representation of first sensor data 604 obtained by the first sensor 208(1) and second sensor data 605 obtained by the second sensor 208(2), all during some period 606, extending between t=0 and t=9. Profiles of the sensor data 604 and 605 may vary at different times between t=0 and t=9. In some examples, the sampling rates of the sensors 208(1) and 208(2) may vary between t=0 and t=9. The diagram 600 also includes timestamps 608 and 610 corresponding, respectively, to a user input or a machine-defined beginning and end of a virtual motor exam. For example, after being prompted by the user device 102 to perform the virtual motor exam, the user may, at t=1, select a button on the user device 102 to begin the virtual motor exam. The timestamp 608 may be generated by the user device 102 and associated with the sensor data 604 and 605 at that time. This may correspond to block 302 of FIG. 3.

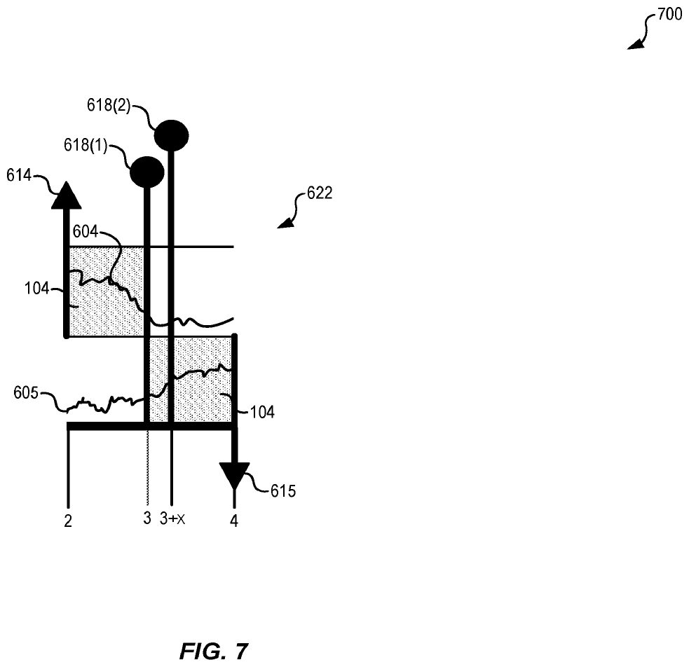

In FIG. 6, the first sensor data 604 obtained from the first sensor 208(1) may be used to determine the first window beginning 614 and the first window end 616. Similarly, the second sensor data 604 obtained from the second sensor 208(2) may be used to determine the second beginning 615 and the second end 617, as described at block 314 of FIG. 3. At this point, there are essentially two context windows, one for each of the two sensors 208(1) and 208(2). User device 102 may analyze the two context windows to identify the context window 612 bounded by the aggregate beginning 618 and the aggregate end 620. In this sense, the context window is different from the context windows 412 and 512 because it has time-shifted. This may result in signal data that is collectively better than just using a context window 412 or 512. FIG. 7 depicts a detailed view of beginning region 622 as it relates to computing the aggregate beginning 618 of the context window 612. The segmenting of the sensor data 604 and 605 may be performed similarly as described with other figures.

FIG. 7 illustrates diagram 700 depicting a detailed view of a beginning region 622 of the diagram 600, according to at least one example. In addition to showing a detailed view, the diagram 700 depicts an iterative process for refining the time associated with the aggregate beginning 618, as depicted at an old time as 618(1) and at a new time as 618(2). The sensor data 604 and 605 is also illustrated in greater detail to represent that the process of determining the aggregate beginning 618 may include one or more iterative evaluations of the sensor data 604 and 605. For example, as part of a first evaluation, the aggregate beginning 618 was identified as 618(1). The process 300 includes blocks 302-312 and may be repeated on a window of time extending between t=2 and t=4 for the sensor data 604 and the sensor data 605 to again identify a potential beginning. These new computations can be used to generate an offset value for the aggregate beginning 618(1) to arrive at the aggregate beginning 618(2). In some examples, this may include picking the middle time between the first beginning 614 and the second beginning 615. In some examples, determining the aggregate beginning 618(2) may include defining the aggregate beginning 618(2) as the same time as the beginning 614 or 615. In some examples, determining the aggregate beginning 618(2) may include picking the aggregate beginning from among a set of beginnings including the beginning of the context window 614 and the beginnings of the one or more different context windows 612. For example, such picking may be based on the sensor type used to define the context window and/or the exam type. This may be possible because data output by certain sensors may be more reliable for determining the beginning of the context window than others and/or certain exam types may have better defined beginnings (and ends) than others exam types. In some examples, determining the aggregate beginning may include averaging the times for the beginning and the other beginnings.

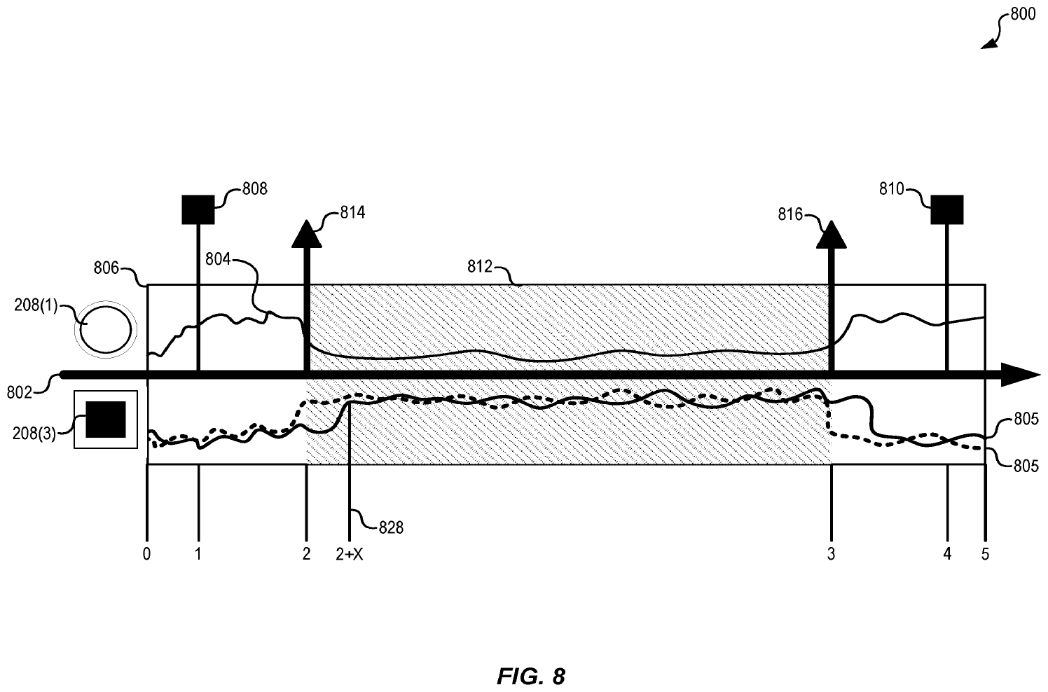

FIG. 8 illustrates a diagram 800 including a first example sensor 208(1) from the user device 102 and a second sensor 208(3) from a different device, according to at least one example. For example, the second sensor 208(3) may be one of the external sensors 130. The diagram 800 is an example of using a first sensor 208(1) to generate a context window, time-aligning data of a second sensor 208(3) with that of the first sensor 208(1), and using the context window to segment data from the first sensor 208(1) and the second sensor 208(3). The diagram 800 includes a timeline 802 and a representation of first sensor data 804 obtained by the first sensor 208(1) and second sensor data 805 obtained by the second sensor 208(3), all during some period 806, extending between t=0 and t=5. Profiles of the sensor data 804 and 805 may vary at different times between t=0 and t=5. In some examples, the sampling rates of the sensors 208(1) and 208(3) may vary between t=0 and t=5. The diagram 800 also includes timestamps 808 and 810 corresponding, respectively, to a user input or a machine-defined beginning and end of a virtual motor exam. For example, after being prompted by the user device 102 to perform the virtual motor exam, the user may, at t=1, select a button on the user device 102 to begin the virtual motor exam. The timestamp 808 may be generated by the user device 102 and associated with the sensor data 804. Because the sensor data 805 is obtained by a different device, the sensor data 805 may be streamed, shared, or otherwise sent to the user device 102. The sensor data 804 and 805 may be out of alignment because it was captured using devices and/or sensors having different internal clocks.

The process of generating the context window 812 may be performed as described elsewhere herein. Once the context window 812 has been defined, the window beginning 814, the window end 816, the timestamps 808 and 810, and/or any other points of the first sensor data 804 may be compared to the second sensor data 805 based on the sensor type of the second sensor 208(3). This may reveal an offset 828 (e.g., “X”) between the first sensor data 804 and the second sensor data 805. To account for the offset 828, the second sensor data 805 may be time-shifted at least until the identified window beginnings match, as illustrated by the dashed version of the second sensor data 805. Once the context window 812 has been defined, it can be used, as described elsewhere herein, to segment sensor data output by the first sensor 208(1), the second sensor 208(3), and any other sensor.

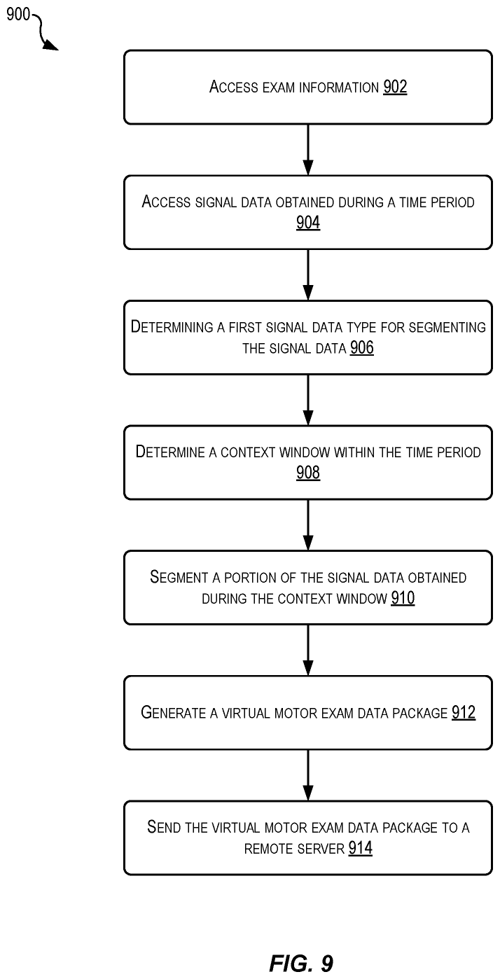

FIG. 9 illustrates an example flowchart illustrating the process 900 relating to implementing techniques relating to segmenting sensor data of virtual motor exams, according to at least one example. The process 900 is performed by the user device 102 (FIG. 1). The process 900 in particular corresponds to various approaches for segmenting sensor data, according to various examples. In some examples, the user device 102 is a wearable user device such as a watch or other device described herein.

The process 900 begins at block 902 by the user device 102 accessing exam information. The exam information may identify: (i) a first timing indicator (e.g., a first timestamp) associated with a first time, (ii) a second timing indicator (e.g., a second timestamp) associated with a second time, and (iii) a virtual motor exam type of a virtual motor exam. In some examples, each of the first and second timing indicators comprises a data tag including a corresponding timestamp. The virtual motor exam may include a series of tasks to evaluate motor function of a wearer of the user device.

In some examples, the process 900 may further include the user device 102 generating the exam information as part of conducting the virtual motor exam during the time period. In some examples, the process 900 may further include the user device 102 receiving a first user input indicating a beginning of the virtual motor exam, generating the first timing indicator responsive to receiving the first user input and based on the first user input, receiving a second user input indicating an end of the virtual motor exam, and generating the second timing indicator responsive to receiving the second user input and based on the second user input.

At block 904, the process 900 includes the user device 102 accessing signal data obtained during a time period bounded by the first time and the second time. The user device 102 may have obtained the signal data using one or more sensors. In some examples, the signal data may include signal data collected from a plurality of sensors of the user device.

At block 906, the process 900 includes the user device 102 determining a first signal data type for segmenting the signal data. This may be based on the virtual motor exam type identified at the block 902. The signal data type (e.g., the first signal data type) may be defined by the sensor used. For example, an accelerometer may output accelerometer-type data. The first signal data of the first signal data type may have been output by a first sensor of the wearable user device during the time period. The first sensor may include any one of the sensors described herein such as, for example, a gyroscope, an accelerometer, a photoplethysmography (PPG) sensor, a heart rate sensor, etc.

At block 908, the process 900 includes the user device 102 determining a context window. The context window may be determined within the time period. In some examples, determining the context window may include selecting a historical signal profile of the first signal data type. The historical signal profile may be derived from previous occurrences of the virtual motor exam. Determining the context window may also include comparing the first signal data to the historical signal profile to identify a third time corresponding to a beginning of the context window and a fourth time corresponding to an end of the context window. In some examples, the context window may include a beginning and an end. The beginning of the context window may be associated with a third time that is later than the first time and earlier the second time. The end of the context window may be associated with a fourth time that is later than the third time and earlier than the second time. In some examples, comparing the first signal data to the historical signal profile may include accessing a set of evaluation rules associated with the virtual motor exam type, and evaluating the first signal data in accordance with the set of evaluation rules to identify the third time and the fourth time. In some examples, the set of evaluation rules may define, for the virtual motor exam type, signal characteristics indicative of the beginning of the context window and the end of the context window.

In some examples, the beginning and the end of the context window are a first beginning and a first end of the context window. In this example, the process 900 may further include determining, by the user device 102 and based on the virtual motor exam type, a second signal data type for segmenting the signal data. The second signal data of the second signal data type may have been output by a second sensor of the user device during the time period. In this example, block 908 may include accessing a different set of evaluation rules associated with the virtual motor exam type, and evaluating the second signal data in accordance with the different set of evaluation rules to identify a second beginning of the context window and a second end of the context window. In this example, the set of evaluation rules may be associated with the first signal data type and the different set of evaluation rules is associated with the second signal data type.

In some examples, the first beginning may be different than the second beginning. In some examples, the process 900 may further include the user device 102 determining an actual beginning of the context window by performing one or more of selecting the actual beginning based on the earlier occurring of the first beginning or the second beginning, or selecting the actual beginning based a comparison of a first signal difference measured between the first signal data at the first beginning and a corresponding first time in the historical signal profile and a second signal difference measured between the second signal data at the second beginning and a corresponding second time in the historical signal profile.

In some examples, the process 900 may further include the user device 102 determining a third timing indicator associated with the third time and a fourth timing indicator associated with the fourth time, and associating the third and fourth timing indicators with the portion of the signal data.

In some examples, the user device 102 may include determining, based on the virtual motor exam type, a second signal data type for segmenting the signal data. The second signal data of the second signal data type may be output by a second sensor of the user device during the time period. In this example, determining the context window at 908 may further be based at least in part on the second signal data.

At block 910, the process 900 includes the user device 102 segmenting a portion of the signal data received during the context window. In some examples, the portion of the signal data may include at least a portion of the first signal data. In some examples, the portion of the signal data may exclude the first signal data.

At block 912, the process 900 includes the user device 102 generating a virtual motor exam data package. This may be based on the portion of the signal data and the exam information. In some examples, generating the virtual motor exam data package may include generating results of the virtual motor exam that includes the portion of the signal data. In some examples, the process 900 may further include the user device 102 outputting a portion of the result by presenting the portion of the results at a display of the user device or sending the portion of the results to a remote computing device.

At block 914, the process 900 includes the user device 102 sending the virtual motor exam data package to a remote server such as the service provider 204.

In some examples, the process 900 further includes, during a later virtual motor exam of the first virtual motor exam type, adjusting, by the user device 102, an operation of the first sensor based on the context window. In some examples, the operation may include a sampling rate. In this example, adjusting the sampling rate based on the context window may include instructing the first sensor to capture data at a first sampling rate outside the context window, and instructing the first sensor to capture data at a second sampling rate within the context window.

In some examples, the virtual motor exam may be conducted during the time period. In this example, associating the portion of the signal data with the virtual motor exam may include tagging the portion of the signal data with a beginning of the context window and an end of the context window within the time period in which the virtual motor exam is conducted.

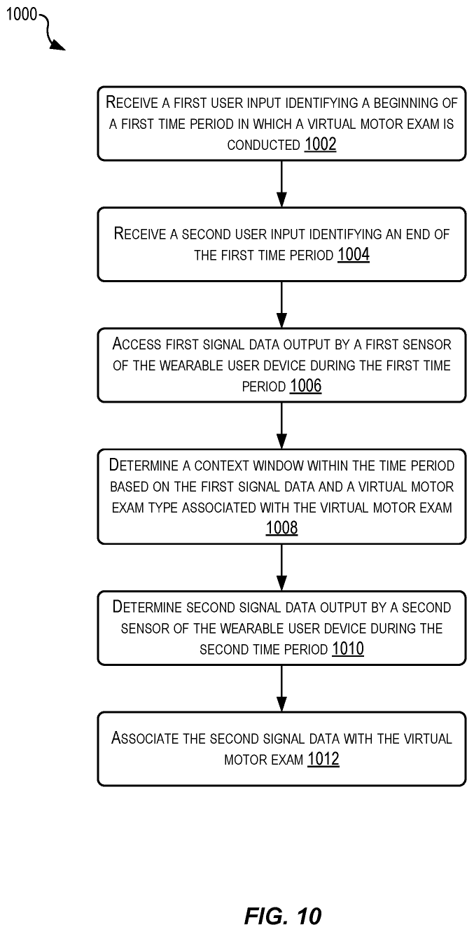

FIG. 10 illustrates an example flowchart illustrating the process 1000 relating to implementing techniques relating to segmenting sensor data of virtual motor exams, according to at least one example. The process 1000 is performed by the user device 102 (FIG. 1). The process 1000 in particular corresponds to various approaches for segmenting sensor data, according to various examples. In some examples, the user device 102 is a wearable user device such as a watch or other device described herein.

The process 1000 begins at block 1002 by the user device 102 receiving, at an input device of the user device 102, a first user input identifying a beginning of a first time period in which a virtual motor exam is conducted. The first input may be received at a graphical user interface, physical button, or at any other location.

At block 1004, the process 1000 includes receiving, at the input device of the user device, a second user input identifying an end of the first time period.

At block 1006, the process 1000 includes accessing, by the user device 102 and based on the virtual motor exam, first signal data output by a first sensor of the user device during the first time period.

At block 1006, the process 1000 includes determining, by the user device 102, a context window within the time period based on the first signal data and a virtual motor exam type associated with the virtual motor exam. The context window may define a second time period that is within the first time period. In some examples, determining the context window within the time period may include accessing a set of evaluation rules associated with the virtual motor exam type, and evaluating the first signal data in accordance with the set of evaluation rules to identify a beginning of the second time period and an end of the second time period. The set of evaluation rules may define, for the virtual motor exam type, signal characteristics indicative of the beginning of the second time period and the end of the second time period.

In some examples, determining the context window defining the second time period may further include accessing a different set of evaluation rules associated with the virtual motor exam type, and evaluating a portion of second signal data obtained during the first time period in accordance with the different set of evaluation rules to identify the beginning of the second time period and the end of the second time period. In some examples, the set of evaluation rules may be associated with a first signal data type of the first signal data and the different set of evaluation rules may be associated with a second signal data type of the second signal data.

At block 1008, the process 1000 includes determining, by the user device 102, second signal data output by a second sensor of the user device during the second time period. In some examples, the first sensor and the second sensor may share a common feature (e.g., each may be capable of tracking some aspect of movement). In some examples, the common feature may be an activity metric. In some examples, the first signal data is distinct from the second signal data.

At block 1010, the process 1000 includes associating, by the user device 102, the second signal data with the virtual motor exam. This may include storing this data in association with each other.

In some examples, the process 1000 may further include the user device 102 segmenting a portion of the first signal data output by the first sensor of the wearable user device during the second time period, and associating the portion of the first signal data with the virtual motor exam.

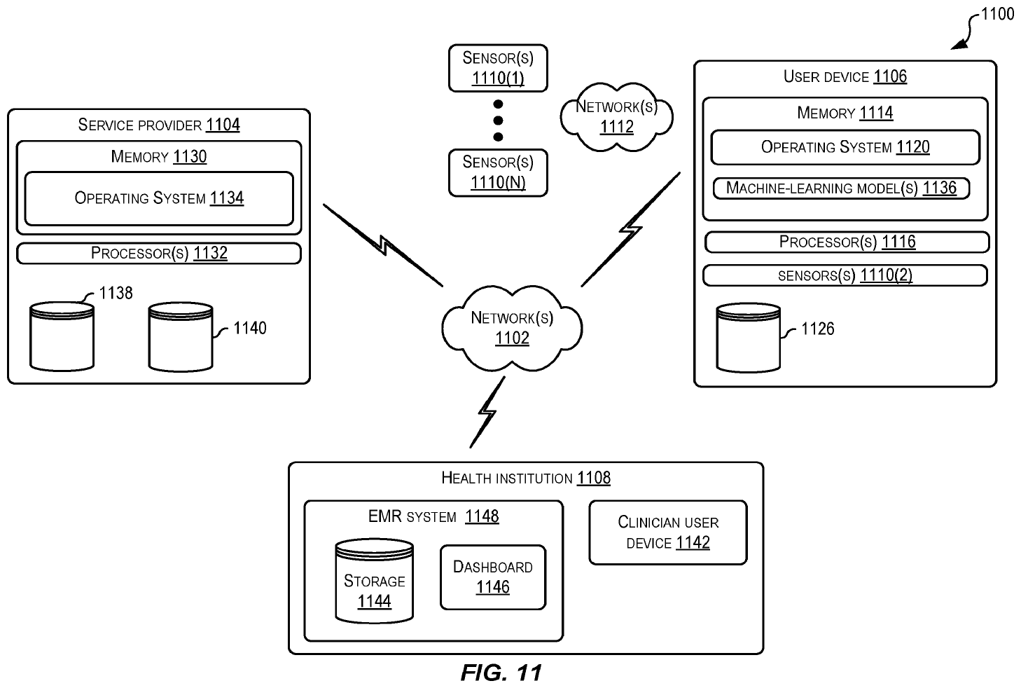

FIG. 11 illustrates an example architecture or environment 1100 configured to implement techniques relating to segmenting sensor data, according to at least one example. For example, the architecture 1100 enables data sharing between the various entities of the architecture, at least some of which may be connected via one or more networks 1102, 1112. For example, the example architecture 1100 may be configured to enable a user device 1106 (e.g., the user device 102), a service provider 1104 (e.g., the service provider 204, sometimes referred to herein as a remote server, service-provider computer, and the like), a health institution 1108, and any other sensors 1110 (e.g., the sensors 116-120 and 130) to share information. In some examples, the service provider 1104, the user device 1106, the health institution 1108, and the sensors 1110(1)-1110(N) may be connected via one or more networks 1102 and/or 1112 (e.g., via Bluetooth, WiFi, the Internet, cellular, or the like). In the architecture 1100, one or more users may utilize a different user device to manage, control, or otherwise utilize the user device 1106 via the one or more networks 1112 (or other networks). Additionally, in some examples, the user device 1106, the service provider 1104, and the sensors 1110 may be configured or otherwise built as a single device such that the functions described with respect to the service provider 1104 may be performed by the user device 1106 and vice versa.

In some examples, the networks 1102, 1112 may include any one or a combination of many different types of networks, such as cable networks, the Internet, wireless networks, cellular networks, satellite networks, other private and/or public networks, or any combination thereof. While the illustrated example represents the user device 1106 accessing the service provider 1104 via the networks 1102, the described techniques may equally apply in instances where the user device 1106 interacts with the service provider 1104 over a landline phone, via a kiosk, or in any other manner. It is also noted that the described techniques may apply in other client/server arrangements (e.g., set-top boxes), as well as in non-client/server arrangements (e.g., locally stored applications, peer-to-peer configurations).

As noted above, the user device 1106 may be configured to collect and/or manage user activity data potentially received from the sensors 1110. In some examples, the user device 1106 may be configured to provide health, fitness, activity, and/or medical data of the user to a third- or first-party application (e.g., the service provider 1104). In turn, this data may be used by the service provider 1104 in implementing techniques described herein.

The user device 1106 may be any type of computing device, such as, but not limited to, a mobile phone, a smartphone, a personal digital assistant (PDA), a wearable device (e.g., ring, watch, necklace, sticker, belt, shoe, shoe attachment, belt-clipped device) an implantable device, or the like. In some examples, the user device 1106 may be in communication with the service provider 1104; the sensors 1110; and/or the health institution via the networks 1102, 1112; or via other network connections.

The sensors 1110 may be standalone sensors or may be incorporated into one or more devices. In some examples, the sensors 1110 may collect sensor data that is shared with the user device 1106 and related to implementing the techniques described herein. For example, the user device 1106 may be a primary user device 1106 (e.g., a smartphone) and the sensors 1110 may be sensor devices that are external from the user device 1106 and can share sensor data with the user device 1106. For example, external sensors 1110 may share information with the user device 1106 via the network 1112 (e.g., via Bluetooth or other near-field communication protocol). In some examples, the external sensors 1110 include network radios that allow them to communicate with the user device 1106 and/or the service provider 1104. The user device 1106 may include one or more applications for managing the remote sensors 1110. This may enable pairing with the sensors 1110, data reporting frequencies, data processing of the data from the sensors 1110, data alignment, and the like.

The sensors 1110 may be attached to various parts of a human body (e.g., feet, legs, torso, arms, hands, neck, head, eyes) to collect various types of information, such as activity data, movement data, or heart rate data. The sensors 1110 may include accelerometers, respiration sensors, gyroscopes, PPG sensors, pulse oximeters, electrocardiogram (ECG) sensors, electromyography (EMG) sensors, electroencephalography (EEG) sensors, global positioning system (GPS) sensors, auditory sensors (e.g., microphones), ambient light sensors, barometric altimeters, electrical and optical heart rate sensors, and any other suitable sensor designed to obtain physiological data, physical condition data, and/or movement data of a patient.

In one illustrative configuration, the user device 1106 may include at least one memory 1114 and one or more processing units (or processor(s)) 1116. The processor(s) 1116 may be implemented as appropriate in hardware, computer-executable instructions, firmware, or combinations thereof. Computer-executable instruction or firmware implementations of the processor(s) 1116 may include computer-executable or machine-executable instructions written in any suitable programming language to perform the various functions described. The user device 1106 may also include geo-location devices (e.g., a GPS device or the like) for providing and/or recording geographic location information associated with the user device 1106. The user device 1106 also includes one or more sensors 1110(2), which may be of the same type as those described with respect to the sensors 1110.

Depending on the configuration and type of the user device 1106, the memory 1114 may be volatile (such as random-access memory (RAM)) and/or non-volatile (such as read-only memory (ROM), flash memory). While the volatile memory described herein may be referred to as RAM, any volatile memory that would not maintain data stored therein once unplugged from a host and/or power would be appropriate.

Both the removable and non-removable memory 1114 are examples of non-transitory computer-readable storage media. For example, non-transitory computer-readable storage media may include volatile or non-volatile, removable or non-removable media implemented in any method or technology for storage of information such as computer-readable instructions, data structures, program modules, or other data. The memory 1114 is an example a of non-transitory computer-readable storage media or non-transitory computer-readable storage device. Additional types of computer storage media that may be present in the user device 1106 may include, but are not limited to, PRAM, SRAM, DRAM, RAM, ROM, EEPROM, flash memory or other memory technology, CD-ROM, DVD or other optical storage, magnetic cassettes, magnetic tape, magnetic disk storage or other magnetic storage devices, or any other medium that can be used to store the desired information and that can be accessed by the user device 1106. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media.

Turning to the contents of the memory 1114 in more detail, the memory 1114 may include an operating system 1120 and/or one or more application programs or services for implementing the features disclosed herein. The user device 1106 also includes one or more machine-learning models 1136 representing any suitable predictive model. The machine-learning models 1136 may be utilized by the user device 1106 to determine the context window, as described herein.

The service provider 1104 may also include a memory 1124 including one or more applications programs or services for implementing the features disclosed herein. In this manner, the techniques described herein may be implemented by any one, or a combination of more than one, of the computing devices (e.g., the user device 1106 and the service provider 1104).

The user device 1106 also includes a datastore that includes one or more databases or the like for storing data such as sensor data 1126 and static data 1128. In some examples, the databases 1126 and 1128 may be accessed via a network service.

The service provider 1104 may also be any type of computing device, such as, but not limited to, a mobile phone, a smartphone, a PDA, a laptop computer, a desktop computer, a thin-client device, a tablet computer, a wearable device, a server computer, or a virtual machine instance. In some examples, the service provider 1104 may be in communication with the user device 1106 and the health institution 1108 via the network 1102 or via other network connections.

In one illustrative configuration, the service provider 1104 may include at least one memory 1130 and one or more processing units (or processor(s)) 1132. The processor(s) 1132 may be implemented as appropriate in hardware, computer-executable instructions, firmware, or combinations thereof. Computer-executable instruction or firmware implementations of the processor(s) 1132 may include computer-executable or machine-executable instructions written in any suitable programming language to perform the various functions described.

The memory 1130 may store program instructions that are loadable and executable on the processor(s) 1132, as well as data generated during the execution of these programs. Depending on the configuration and type of service provider 1104, the memory 1130 may be volatile (such as RAM) and/or non-volatile (such as ROM, flash memory). While the volatile memory described herein may be referred to as RAM, any volatile memory that would not maintain data stored therein once unplugged from a host and/or power would be appropriate. Both the removable and non-removable memory 1130 are additional examples of non-transitory computer-readable storage media.

Turning to the contents of the memory 1130 in more detail, the memory 1130 may include an operating system 1134 and/or one or more application programs or services for implementing the features disclosed herein.

The service provider 1104 also includes a datastore that includes one or more databases or the like for storing data, such as sensor data 1138 and static data 1140. In some examples, the databases 1138 and 1140 may be accessed via a network service.

Turning now to the health institution 1108, while depicted as a single entity, the health institution 1108 may represent multiple health institutions. The health institution 1108 includes an EMR system 1148, which is accessed via a dashboard 1146 (e.g., by a user using a clinician user device 1142). In some examples, the EMR system 1148 may include a record storage 1144 and a dashboard 1146. The record storage 1144 may be used to store health records of patients associated with the health institution 1108. The dashboard 1146 may be used to read and write the records in the record storage 1144. In some examples, the dashboard 1146 is used by a clinician to manage disease progression for a patient population including a patient who operates the user device 102. The clinician may operate the clinician user device 1142 to interact with the dashboard 1146 to view results of virtual motor exams on a patient-by-patient basis, on a population of patient basis, etc. In some examples, the clinician may use the dashboard 1146 to “push” an exam to the user device 102.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions, and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computing systems accessing stored software that programs or configures the computing system from a general-purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied-for example, blocks can be reordered, combined, and/or broken into sub-blocks. Certain blocks or processes can be performed in parallel.

Conditional language used herein, such as among others, “can,” “could,” “might,” “may,” “e.g.,” and the like, unless specifically stated otherwise or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements and/or steps are included or are to be performed in any particular example.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Use herein of the word “or” is intended to cover inclusive and exclusive OR conditions. In other words, “A or B or C” includes any or all of the following alternative combinations as appropriate for a particular usage: A alone; B alone; C alone; A and B only; A and C only; B and C only; and all three of A and B and C.

The use of the terms “a,” “an,” and “the” and similar referents in the context of describing the disclosed examples (especially in the context of the following claims) are to be construed to cover both the singular and the plural, unless otherwise indicated herein or clearly contradicted by context. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may in practice be based on additional conditions or values beyond those recited. Headings, lists, and numbering included herein are for ease of explanation only and are not meant to be limiting.

The various features and processes described above may be used independently of one another or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

All references, including publications, patent applications, and patents, cited herein are hereby incorporated by reference to the same extent as if each reference were individually and specifically indicated to be incorporated by reference and were set forth in its entirety herein.

1. A computer-implemented method, comprising:
accessing, by a wearable user device, exam information identifying: (i) a first timing indicator associated with a first time, (ii) a second timing indicator associated with a second time, and (iii) a virtual motor exam type of a virtual motor exam;
accessing, by the wearable user device, signal data obtained by the wearable user device during a time period bounded by the first time and the second time;
determining, by the wearable user device and based on the virtual motor exam type, a first signal data type for segmenting the signal data, the first signal data of the first signal data type being output by a first sensor of the wearable user device during the time period;
determining, by the wearable user device, a context window within the time period by at least:
selecting a historical signal profile of the first signal data type, the historical signal profile derived from previous occurrences of the virtual motor exam; and
comparing the first signal data to the historical signal profile to identify a third time corresponding to a beginning of the context window and a fourth time corresponding to an end of the context window of the context window;
segmenting, by the wearable user device, a portion of the signal data received during the context window; and
generating, by the wearable user device, a virtual motor exam data package based on the portion of the signal data and the exam information.

2. The computer-implemented method of claim 1, further comprising, during a later virtual motor exam of the virtual motor exam type, adjusting, by the wearable user device, an operation of the first sensor based on the context window.

3. The computer-implemented method of claim 2, wherein the operation comprises a sampling rate, and wherein adjusting the sampling rate based on the context window comprises instructing the first sensor to capture data at a first sampling rate outside the context window, and instructing the first sensor to capture data at a second sampling rate within the context window.

4. The computer-implemented method of claim 1, wherein comparing the first signal data to the historical signal profile further comprises:
accessing a set of evaluation rules associated with the virtual motor exam type; and
evaluating the first signal data in accordance with the set of evaluation rules to identify the third time and the fourth time.

5. The computer-implemented method of claim 4, wherein the set of evaluation rules defines, for the virtual motor exam type, signal characteristics indicative of the beginning of the context window and the end of the context window.

6. The computer-implemented method of claim 4, wherein the beginning and the end of the context window are a first beginning and a first end of the context window, the method further comprising determining, by the wearable user device and based on the virtual motor exam type, a second signal data type for segmenting the signal data, second signal data of the second signal data type being output by a second sensor of the wearable user device during the time period, and wherein determining the context window within the time period further comprises:
accessing a different set of evaluation rules associated with the virtual motor exam type; and
evaluating the second signal data in accordance with the different set of evaluation rules to identify a second beginning of the context window and a second end of the context window.

7. The computer-implemented method of claim 6, wherein the set of evaluation rules is associated with the first signal data type and the different set of evaluation rules is associated with the second signal data type.

8. The computer-implemented method of claim 6, wherein the first beginning is different than the second beginning, and wherein the method further comprises determining an actual beginning of the context window by performing one or more of:
selecting the actual beginning based on the earlier occurring of the first beginning or the second beginning; and
selecting the actual beginning based a comparison of a first signal difference measured between the first signal data at the first beginning and a corresponding first time in the historical signal profile, and a second signal difference measured between the second signal data at the second beginning and a corresponding second time in the historical signal profile.

9. The computer-implemented method of claim 1, wherein the context window comprises a beginning and an end, and wherein the beginning of the context window is associated with a third time that is later than the first time and earlier the second time, and wherein the end of the context window is associated with a fourth time that is later than the third time and earlier than the second time.

10. The computer-implemented method of claim 9, further comprising:
determining a third timing indicator associated with the third time and a fourth timing indicator associated with the fourth time; and
associating the third and fourth timing indicators with the portion of the signal data.

11. The computer-implemented method of claim 1, further comprising generating the exam information as part of conducting the virtual motor exam during the time period.

12. The computer-implemented method of claim 1, further comprising:
receiving, at the wearable user device, a first user input indicating a beginning of the virtual motor exam;
generating the first timing indicator responsive to receiving the first user input and based on the first user input;
receiving, at the wearable user device, a second user input indicating an end of the virtual motor exam; and
generating the second timing indicator responsive to receiving the second user input and based on the second user input.

13. The computer-implemented method of claim 1, wherein each of the first and second timing indicators comprises a data tag including a corresponding timestamp.

14. The computer-implemented method of claim 1, wherein the signal data comprises signal data collected from a plurality of sensors of the wearable user device.

15. The computer-implemented method of claim 1, further comprising determining, by the wearable user device and based on the virtual motor exam type, a second signal data type for segmenting the signal data, second signal data of the second signal data type being output by a second sensor of the wearable user device during the time period.

16. The computer-implemented method of claim 15, wherein determining the context window is further based at least in part on the second signal data.

17. The computer-implemented method of claim 1, wherein the portion of the signal data comprises at least a portion of the first signal data.

18. The computer-implemented method of claim 1, wherein the portion of the signal data excludes the first signal data.

19. The computer-implemented method of claim 1, wherein the first sensor comprises at least one of a gyroscope, an accelerometer, a photoplethysmography sensor, or a heart rate sensor.

20. The computer-implemented method of claim 1, wherein the virtual motor exam comprises a series of tasks to evaluate motor function of a wearer of the wearable user device.

21. The computer-implemented method of claim 1, further comprising:
generating results of the virtual motor exam that include the portion of the signal data; and
outputting a portion of the result, wherein outputting the portion of the results comprises at least one of presenting the portion of the results at a display of the wearable user device or sending the portion of the results to a remote computing device.

22. The computer-implemented method of claim 1, wherein the virtual motor exam is conducted during the time period, and wherein associating the portion of the signal data with the virtual motor exam comprises tagging the portion of the signal data with a beginning of the context window and an end of the context window within the time period in which the virtual motor exam is conducted.

23. A computer-readable medium comprising processor-executable instructions that, when executed by one or more processors of a wearable device, cause the wearable device to perform operations comprising:
accessing, by the wearable user device, exam information identifying: (i) a first timing indicator associated with a first time, (ii) a second timing indicator associated with a second time, and (iii) a virtual motor exam type of a virtual motor exam;
accessing, by the wearable user device, signal data obtained by the wearable user device during a time period bounded by the first time and the second time;
determining, by the wearable user device and based on the virtual motor exam type, a first signal data type for segmenting the signal data, the first signal data of the first signal data type being output by a first sensor of the wearable user device during the time period;
determining, by the wearable user device, a context window within the time period by at least:
selecting a historical signal profile of the first signal data type, the historical signal profile derived from previous occurrences of the virtual motor exam; and
comparing the first signal data to the historical signal profile to identify a third time corresponding to a beginning of the context window and a fourth time corresponding to an end of the context window of the context window;
segmenting, by the wearable user device, a portion of the signal data received during the context window; and
generating, by the wearable user device, a virtual motor exam data package based on the portion of the signal data and the exam information.

24. A wearable user device comprising:
configured to store computer-executable instructions;
one or more processors configured to access the memory and execute the computer-executable instructions to at least:
access exam information identifying: (i) a first timing indicator associated with a first time, (ii) a second timing indicator associated with a second time, and (iii) a virtual motor exam type of a virtual motor exam;
access signal data obtained by the wearable user device during a time period bounded by the first time and the second time;
determine, based on the virtual motor exam type, a first signal data type for segmenting the signal data, the first signal data of the first signal data type being output by a first sensor of the wearable user device during the time period;
determine a context window within the time period by at least:
selecting a historical signal profile of the first signal data type, the historical signal profile derived from previous occurrences of the virtual motor exam; and
comparing the first signal data to the historical signal profile to identify a third time corresponding to a beginning of the context window and a fourth time corresponding to an end of the context window of the context window;
segmenting a portion of the signal data received during the context window; and
generating a virtual motor exam data package based on the portion of the signal data and the exam information.

25. A computer-implemented method, comprising:
receiving, at an input device of a wearable user device, a first user input identifying a beginning of a first time period in which a virtual motor exam is conducted;
receiving, at the input device of the wearable user device, a second user input identifying an end of the first time period;
accessing, by the wearable user device and based on the virtual motor exam, first signal data output by a first sensor of the wearable user device during the first time period;
determining, by the wearable user device, a context window within the first time period based on the first signal data and a virtual motor exam type associated with the virtual motor exam, the context window defining a second time period that is within the first time period;
determining, by the wearable user device, second signal data output by a second sensor of the wearable user device during the second time period; and
associating, by the wearable user device, the second signal data with the virtual motor exam.

26. The computer-implemented method of claim 25, wherein the first sensor and the second sensor share a common feature.

27. The computer-implemented method of claim 26, wherein the common feature comprises an activity metric.

28. The computer-implemented method of claim 25, wherein the first signal data is distinct from the second signal data.

29. The computer-implemented method of claim 25, further comprising segmenting a portion of the first signal data output by the first sensor of the wearable user device during the second time period; and
associating the portion of the first signal data with the virtual motor exam.

30. The computer-implemented method of claim 25, wherein determining the context window within the first time period comprises:
accessing a set of evaluation rules associated with the virtual motor exam type; and
evaluating the first signal data in accordance with the set of evaluation rules to identify a beginning of the second time period and an end of the second time period.

31. The computer-implemented method of claim 30, wherein the set of evaluation rules defines, for the virtual motor exam type, signal characteristics indicative of the beginning of the second time period and the end of the second time period.

32. The computer-implemented method of claim 30, wherein determining the context window defining the second time period further comprises:
accessing a different set of evaluation rules associated with the virtual motor exam type; and
evaluating a portion of second signal data obtained during the first time period in accordance with the different set of evaluation rules to identify the beginning of the second time period and the end of the second time period, wherein the set of evaluation rules is associated with a first signal data type of the first signal data and the different set of evaluation rules is associated with a second signal data type of the second signal data.

33. (canceled)

34. (canceled)

35. The computer-implemented method of claim 25, wherein the context window corresponds to a time when a user performs the virtual motor exam.

36. A computer-readable medium comprising processor-executable instructions that, when executed by one or more processors of a wearable user device, cause the wearable user device to perform operations comprising:
receiving, at an input device of the wearable user device, a first user input identifying a beginning of a first time period in which a virtual motor exam is conducted;
receiving, at the input device of the wearable user device, a second user input identifying an end of the first time period;
accessing, by the wearable user device and based on the virtual motor exam, first signal data output by a first sensor of the wearable user device during the first time period;
determining, by the wearable user device, a context window within the first time period based on the first signal data and a virtual motor exam type associated with the virtual motor exam, the context window defining a second time period that is within the first time period;
determining, by the wearable user device, second signal data output by a second sensor of the wearable user device during the second time period; and
associating, by the wearable user device, the second signal data with the virtual motor exam.

37. (canceled)
